(19) United States
(12) Patent Application Publication
(10) Pub. No.: US 2016/0089039 A1
(43) Pub. Date: Mar. 31, 2016

(54) Liner System for Blood Pressure Cuffs and Corresponding Methods

(71) Applicant: Medline Industries, Inc., Mundelein, IL (US)

(72) Inventors: Michael Turturro, Alrington Heights, IL (US); Andy Mills, Highland Park, IL (US); Mary M. Peplinski, Mundelein, IL (US); Susan Gibbons, Barrington, IL (US); Francesca Oliver, Chicago, IL (US); Ed Drower, Chicago, IL (US); Megan Henken, Wauconda, IL (US); Michael Kobida, Lake Barrington, IL (US)

(21) Appl. No.: 14/500,662

(22) Filed: Sep. 29, 2014

(51) Int. Cl. A61B 5/021; B32B 7/06; B32B 7/12

(52) U.S. Cl. CPC A61B 5/02141; B32B 7/12; B32B 7/06; B32B 2535/00

(57) ABSTRACT

A liner system (500) for use with a blood pressure cuff (500) includes a plurality of layers (503,504,505,506) of material arranged in a stack (502). Each layer of material can be peelable from other layers of material such that an uppermost layer of the stack can be peeled from the stack after use to reveal a next uppermost layer of the stack. Each layer of material can include a tab (517,518,519,520) to facilitate peeling the each layer of material from the stack. The tabs can be staggered from other tabs of the stack. The lowermost layer of the stack can include a fixative (1228) to attach the stack to an inner surface (202) of the blood pressure cuff. The liner system prevents the cross-contamination between patients.

### BACKGROUND

1. Technical Field

This disclosure relates generally to cuffs, and more particularly to blood pressure cuffs.

2. Background Art

Healthcare services providers generally take blood pressure readings by placing a cuff about a person's arm. Air is used to inflate the cuff to temporarily restrict blood flow. Once the cuff is deflated, the healthcare services provider monitors a person's pulse with a stethoscope. The healthcare services provider can then calculate the person's systolic and diastolic blood pressure. The process can be automated, with machines performing the operations otherwise performed by the healthcare services provider. Automated processes sometimes determine the systolic and diastolic blood pressure via air pressure pulsations delivered to the cuff.

When a blood pressure cuff is used across patients, i.e., to measure the blood pressure of a first person, then another, and then another, etc., the cuff itself can act as a vector for pathogens, microbes, viruses, and bacteria. In effect, the blood pressure cuff can be responsible for cross-contamination across patients.

One solution to the cross-contamination problem is to provide “single use” blood pressure cuffs. Such cuffs are used only on a single patient, and are then discarded. The problem with this approach is that disposable cuffs are seldom biodegradable, and thus remain in landfills for long periods of time. Moreover, it is costly, as new cuffs must be purchased for each new patient. It would be advantageous to have an improved solution that prevents cross-contamination, yet that is “greener” and less costly.

### BRIEF DESCRIPTION OF THE DRAWINGS

FIG. 1 illustrates a perspective view of one explanatory device employing an explanatory liner system in accordance with one or more embodiments of the disclosure.

FIG. 2 illustrates a front elevation view of one explanatory device employing an explanatory liner system in accordance with one or more embodiments of the disclosure.

FIG. 3 illustrates a rear elevation view of one explanatory device in accordance with one or more embodiments of the disclosure.

FIG. 4 illustrates another front elevation view of one explanatory device employing an explanatory liner system in accordance with one or more embodiments of the disclosure.

FIG. 5 illustrates yet another front elevation view of yet another explanatory device employing an explanatory liner system in accordance with one or more embodiments of the disclosure.

FIG. 6 illustrates yet another front elevation view of yet another explanatory device employing an explanatory liner system in accordance with one or more embodiments of the disclosure.

FIG. 7 illustrates another front elevation view of another explanatory device employing another explanatory liner system in accordance with one or more embodiments of the disclosure.

FIG. 8 illustrates another front elevation view of another explanatory device employing an explanatory liner system in accordance with one or more embodiments of the disclosure.

FIG. 9 illustrates another front elevation view of another explanatory device employing an explanatory liner system in accordance with one or more embodiments of the disclosure.

FIG. 10 illustrates yet another front elevation view of yet another explanatory device employing an explanatory liner system in accordance with one or more embodiments of the disclosure.

FIG. 11 illustrates another explanatory device employing an explanatory liner system in accordance with one or more embodiments of the disclosure.

FIG. 12 illustrates an explanatory system in accordance with one or more embodiments of the disclosure.

FIG. 13 illustrates another explanatory system in accordance with one or more embodiments of the disclosure.

FIG. 14 illustrates an explanatory liner stack in accordance with one or more embodiments of the disclosure.

FIG. 15 illustrates one explanatory method for manufacturing a liner stack in accordance with one or more embodiments of the disclosure.

FIG. 16 illustrates another explanatory method of manufacturing a liner stack in accordance with one or more embodiments of the disclosure.

FIG. 17 illustrates an explanatory system configured in accordance with one or more embodiments of the disclosure in use.

FIG. 18 illustrates an explanatory method of using a system in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

The apparatus components and method steps described below have been represented—where appropriate—by conventional symbols in the drawings, showing only those specific details that are pertinent to understanding the embodiments of the present disclosure so as not to obscure the disclosure with details that will be readily apparent to those of ordinary skill in the art having the benefit of the description herein. It is expected that one of ordinary skill, notwithstanding possibly significant effort and many design choices motivated by, for example, available time, current technology, and economic considerations, when guided by the concepts and principles disclosed herein will be readily capable of generating embodiments of the disclosure with minimal experimentation.

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide a liner system for use with a blood pressure cuff. In one embodiment, the liner system includes a plurality of layers of material. The layers can be arranged in a stack. In one embodiment, the layers are disposable after use. In another embodiment, the layers are reusable after cleaning and/or sanitation.

Each layer of material can be peelable from other layers of material such that an uppermost layer of the stack can be peeled from the stack to reveal a next uppermost layer of the stack. In one embodiment, the stack can be attached —adhesively or otherwise—to the interior side of a blood pressure cuff. A healthcare services provider can then, after using the blood pressure cuff on a patient, peel the uppermost layer of the stack from the stack to reveal the next uppermost layer of the stack. Thus, any contaminants present on the uppermost layer as a result of using the blood pressure cuff are easily and quickly removed, thereby leaving a sterile next uppermost layer for use with the next patient.

In one embodiment, each layer includes a tab to facilitate peeling each layer of material from the stack. In one embodiment, each tab is staggered from other tabs of the stack so that it can easily be grasped. In other embodiments, the tabs are placed atop each other for a more compact arrangement. In one embodiment, the lowermost layer of the stack includes a fixative to attach the stack to an inner surface of the blood pressure cuff. The fixative can be a hook and loop fastener, adhesive, or other fixatives. In one embodiment, once the layers are depleted the blood pressure cuff can be discarded. In another embodiment, another stack can be attached to the blood pressure cuff so that the process can start over.

In one embodiment, the layers cover both sides of the cuff. In another embodiment, the layers only cover a single side of the cuff. Prior to using the cuff on a new patient, a healthcare services provider can peel away the uppermost layer to reveal a fresh, clean surface underneath. The removed layer can be discarded, thereby eliminating the need to discard the entire cuff. This reduces waste, and is less expensive than prior art solutions.

In one or more embodiments, each layer can be numbered or otherwise marked so that the healthcare services provider is informed as to how many layers are remaining and when either the cuff will need to be discarded or a new stack will need to be obtained. In other embodiments, the layers can be color coded to provide this information.

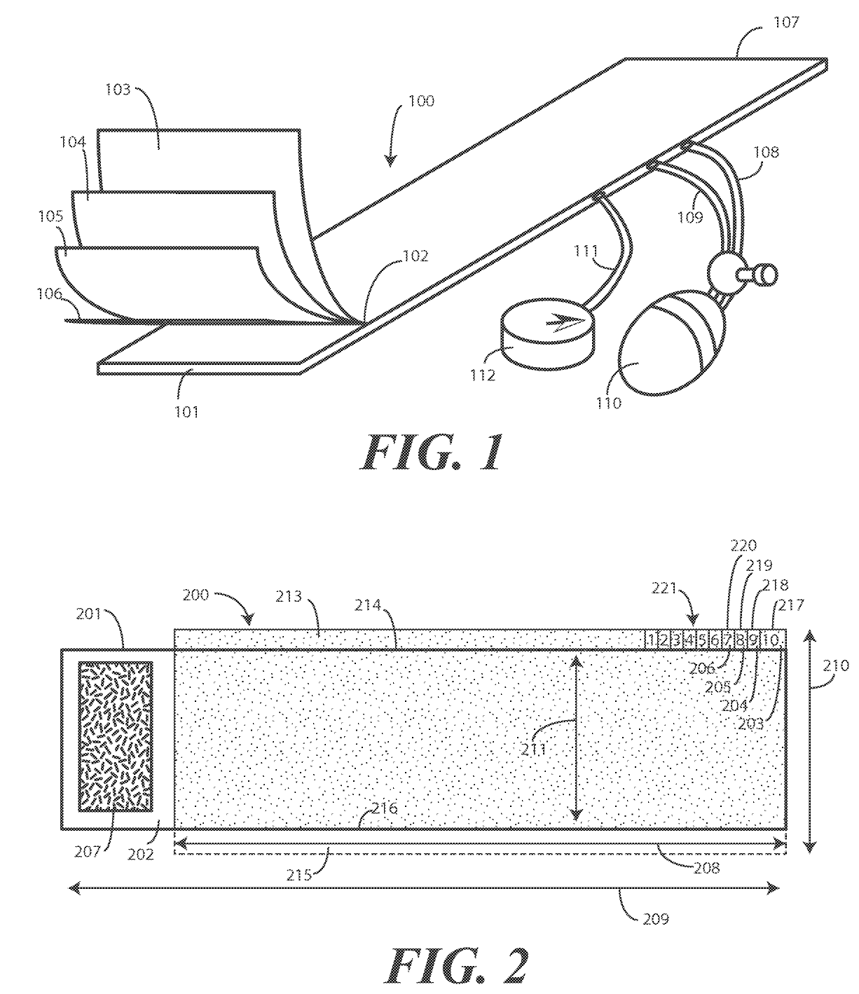

Turning now to FIG. 1, illustrated therein is one explanatory liner system 100 being used with a blood pressure cuff 101. The blood pressure cuff 101 is configured so as to be wrapped about the arm of a patient. In one embodiment, the blood pressure cuff 101 is configured as an elongated, flexible strap that is selectively inflatable. In this illustrative embodiment, the blood pressure cuff 101 is rectangular. However, it will be readily understood by those of ordinary skill in the art having the benefit of this disclosure that the blood pressure cuff 101 could take other configurations as well. In one embodiment, the blood pressure cuff 101 is about twenty inches in length, and is between five and six inches wide.

When wrapped about a patient's arm, the ends of the blood pressure cuff 101 overlap. As will be shown in subsequent figures, hook and loop or other fasteners can be selectively placed along the interior or exterior of the blood pressure cuff 101 to secure it about a patient's arm. An inflatable bladder is disposed within the blood pressure cuff 101. In one embodiment, one or more tubes 108 couple the blood pressure cuff 101 to an inflation pump 110. Other tubing 111 can couple the blood pressure cuff 101 to a sphygmomanometer 112.

The liner system 100 includes a plurality of layers 103,104,105,106 of material, with each layer of material arranged in a stack 102. Each layer 103,104,105,106 of material is peelable from other layers of material such that an uppermost layer, i.e., layer 103, of the stack 102 can be peeled from the stack 102 after use to reveal a next uppermost layer of the stack, i.e., layer 104. Accordingly, the various layers 103,104,105,106 can selectively be peeled away following each use of the blood pressure cuff to prevent patient-to-patient cross-contamination.

In one embodiment, each layer 103,104,105,106 is disposable and is manufactured from plastic. For example, in one embodiment the layers 103,104,105,106 are manufactured from plastic and have a polyester coating so as to feel similar to a traditional cuff. In other embodiments, each layer 103,104,105,106 is reusable. For example, the layers 103,104,105,106 could be attached to each other by a hook and loop fastener. When a first layer is removed, it could be washed and sterilized, and then reattached to the stack 102.

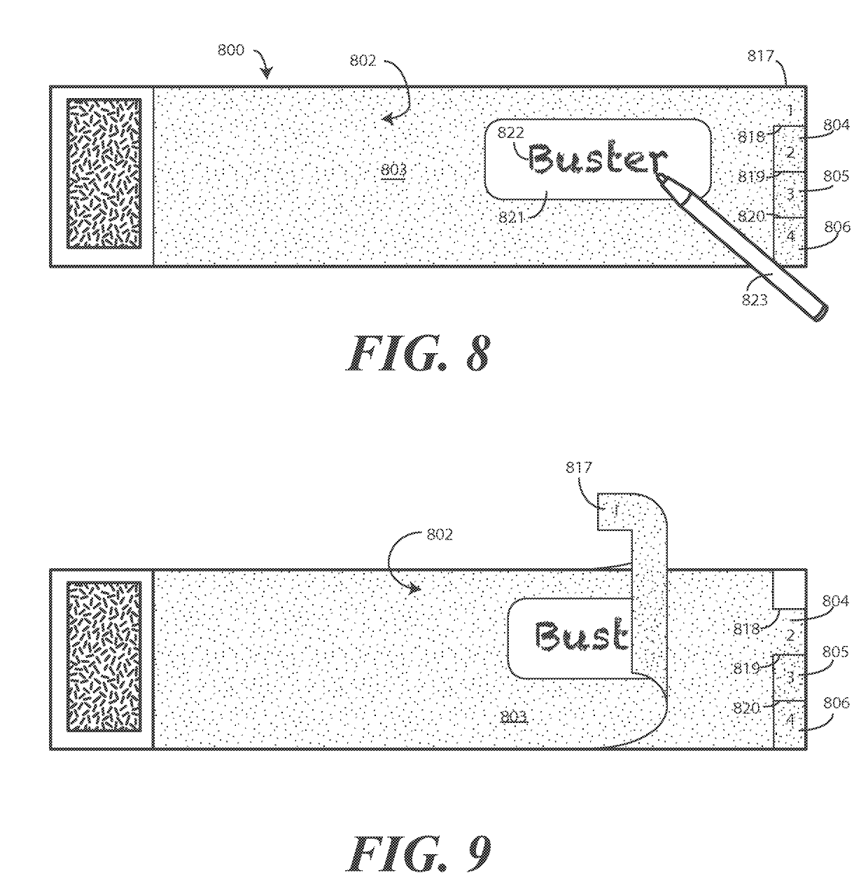

In one embodiment, as will be shown in more detail below with reference to subsequent figures, such as FIGS. 7 and 8, the layers 103,104,105,106 extend beyond the edge 107 of the cuff to protect against unwanted contamination. In one or more embodiments, as will be shown below with reference to FIG. 14, each layer 103,104,105,106 further includes an extra flap to cover the first few inches of tubing 108,109 of the blood pressure cuff 101 to protect against unwanted contamination. The layers 103,104,105,106 are designed to cover inner surface of the blood pressure cuff 101, i.e., those portions that come into contact with the patient when the blood pressure cuff 101 is in use.

In one embodiment, the blood pressure cuff 101 is disposable. In such an embodiment, once the layers 103,104,105,106 have been removed, the healthcare services provider will dispose of the blood pressure cuff. In another embodiment, the blood pressure cuff 101 is a reusable cuff. Once the layers 103,104,105,106 have been removed, the healthcare services provider simply attaches another stack 102 to the interior of the blood pressure cuff 101 to again render it ready for use.

The number of layers 103,104,105,106 in the stack 102 can vary. In one embodiment, the variation in number is a function of application. For example, where the blood pressure cuff 101 is disposable, the number of layers 103,104,105,106 may comprise five or fewer layers, such as between three and five layers. After these three to five layers have been depleted, the blood pressure cuff 101 can be discarded. This reduces waste when compared to prior art cuffs, while ensuring that cross-contamination does not occur.

In another embodiment, where the blood pressure cuff 101 is a reusable cuff, the number of layers 103,104,105,106 may be different. For example, in one embodiment, the number of layers 103,104,105,106 used with a reusable cuff is greater than that used with a disposable cuff. For example, in one embodiment the stack 102 includes a number of layers greater than five, such as ten layers. Accordingly, when each of the layers 103,104,105,106 is removed, a healthcare services provider can purchase another stack 102 and attach it to the blood pressure cuff 101. Stacks of the layers 103,104,105,106 can be sold separately from the blood pressure cuff 101 as an accessory for attachment to the inner surface of the blood pressure cuff 101. Replacement stacks could include different numbers of layers, such as between five and twenty. These layer numbers are illustrative only, as others will be obvious to those of ordinary skill in the art having the benefit of this disclosure.

Experimental testing has shown that the stack 102 of layers 103,104,105,106 does not significantly affect the blood pressure readings taken with the sphygmomanometer 112. For example, in testing where the blood pressure cuff 101 without the stack 102 of layers 103,104,105,106 measured a systolic blood pressure of 105, the blood pressure cuff 101 measured when the layers 103,104,105,106 was present was within one percent of this reading. Similarly, when diastolic blood pressure was measured with the blood pressure cuff 101 alone, the measurement with the layers 103,104,105,106 present was still within one percent.

When an automated process was used, the results were equally good. For example, a systolic measurement of 122 without the layers 103,104,105,106 was within a half of a percent of the measurement with the layers 103,104,105,106. Similarly, a diastolic measurement of 76 without the layers 103,104,105,106 was within 1.5 percent of the measurement with the layers 103,104,105,106.

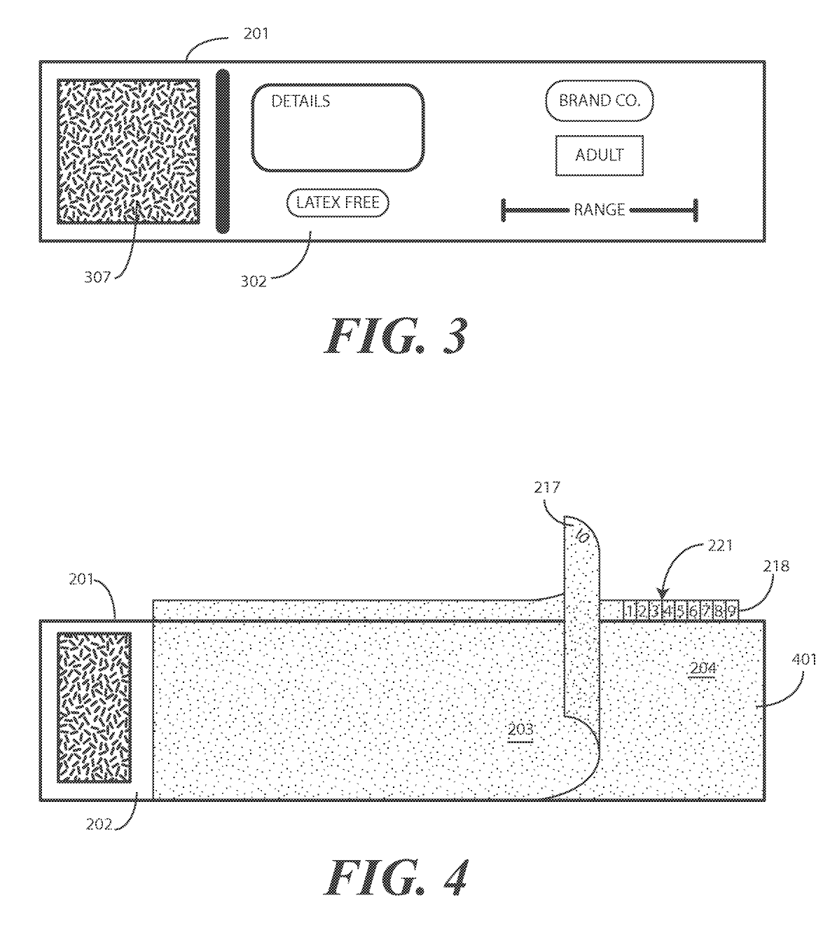

Turning now to FIGS. 2 and 3, illustrated therein is another liner system 200 configured in accordance with one or more embodiments of the disclosure. The liner system 200 of FIG. 2 is also being used with a blood pressure cuff 201. The interior side 202 of the blood pressure cuff 201 is shown in FIG. 2, while the exterior side 302 of the blood pressure cuff 201 is shown in FIG. 3.

As shown, the liner system 200 is attached to the interior side 202 of the blood pressure cuff 201 in this embodiment such that when the blood pressure cuff 201 is wrapped about the arm of a patient, the liners 203,204,205,206 of the liner system 200 prevent the blood pressure cuff 201 from coming in contact with the user's arm. However, as noted above, the layers 203,204,205,206 of the liner system 200 can cover both the interior side 202 and the exterior side 302 of the blood pressure cuff 201 in other embodiments.

In the embodiment of FIGS. 2 and 3, there are ten liners in the stack. As noted above, testing has shown that a larger number of layers are preferable when the blood pressure cuff 201 is reusable, while a lesser number of layers can be advantageous when the blood pressure cuff 201 is disposable.

The dimensions of the liner system 200 can be the same as those of the blood pressure cuff 201 or greater than those of the blood pressure cuff 201. In some instances, they can be less than those of the blood pressure cuff 201 as well. For example, the illustrative blood pressure cuff 201 of FIGS. 2 and 3 includes a first fastener 207 on the interior side 202 of the blood pressure cuff 201, and a second fastener 307 of the exterior side 302 of the blood pressure cuff 201. In one embodiment, the first fastener 207 and the second fastener 307 are hook and loop fasteners. However, other fasteners can be used as well. Where, for example, the blood pressure cuff 201 is disposable, the first fastener 207 and the second fastener 307 can be adhesive fasteners.

Where the interior side 202 includes the first fastener 207, the length of the liner system 200 can be less than the length of the blood pressure cuff 201. In the illustrative embodiment of FIGS. 2 and 3, each layer 203,204,205,206 of material of the liner system 200 defines a major dimension 208 that is less than the length 209 of the blood pressure cuff 201. This allows the portion 212 of the blood pressure cuff 201 upon which the first fastener 207 is disposed to extend from the liner system 200 such that the first fastener 207 is exposed to attach to the second fastener 307. When the blood pressure cuff 201 is wrapped about the patients arm, the portion 212 of the blood pressure cuff 201 upon which the first fastener 207 is disposed will wrap about the exterior 302 of the blood pressure cuff 201 and will not contact the patient's arm. Accordingly, despite the major dimension 208 being less than the length 209 of the blood pressure cuff 201, cross-contamination will still not occur.

In one embodiment, the liner system 200 also defines a minor dimension 210. The minor dimension 210, in one embodiment, is greater than or equal to a width 211 the blood pressure cuff 201 to preclude contact of the blood pressure cuff 201 with a patient's arm. The top 213 of the liner system 200 can extend beyond the top edge 214 of the blood pressure cuff 201. Similarly, the bottom 215 of the liner system 200 can extend beyond the bottom edge 216 of the blood pressure cuff 201 as well. Of course, combinations of the two, i.e., both the top 213 and the bottom 215 extending beyond the edges of the blood pressure cuff 201 can be used as well.

As best shown in FIG. 2, in this illustrative embodiment each layer 203,204,205,206 includes a tab 217,218,219,220 to facilitate peeling the each layer 203,204,205,206 of material from the stack. The tabs 217,218,219,220 of FIG. 2 are disposed along a major side of the blood pressure cuff 201, namely, the top side 214 in this embodiment.

As shown, in one embodiment each tab 217,218,219,220 is staggered from other tabs of the stack to make selectively peeling each layer 203,204,205,206 away from the stack. In other embodiments, each tab 217,218,219,220 is placed atop each other for a more compact arrangement.

In the illustrative embodiment of FIG. 2, the tabs 217,218,219,220 are staggered due to the fact that each layer 203,204,205,206 is slightly shorter than the next along the tabbed portion 221 that extends upward from the upper edge 214 of the blood pressure cuff 201. For example, layer 204 is slightly shorter than layer 203 along the tabbed portion 221, and so forth. Consequently, when tab 217 is pulled away from the stack, as shown in FIG. 4, layer 203 is pulled away from the stack, thereby revealing layer 204, which can also be pulled away by tab 218.

It should be noted that, to provide full coverage across the interior side 202 of the blood pressure cuff 201, layers other than the initially uppermost layer include extension portions that extend past the tab in this embodiment. For example, layer 204, which has a tab 218 that is staggered from tab 217 of layer 203 by making layer 204 shorter along the tabbed portion 221, includes an extension portion 401 that extends beyond the tab 218 so that the interior surface 202 of the blood pressure cuff 201 is completely covered so as to prevent cross contamination between patients. This results in each layer 203,204,205,206 of material having different dimensions than other layers of material in the stack.

In the illustrative embodiment of FIGS. 2-4, each of the tabs 217,218,219,220 is numbered to provide an indication to a healthcare services provider which layer 203,204,205,206 is the uppermost layer. For example, tab 217 has the number “10” disposed thereon, while tab 218 has the number “9” disposed thereon, and so forth. The numbers could be reversed, with the number “1” disposed on tab 217, the number “2” disposed on tab 218, and so forth. In addition to providing an indication of which layer 203,204,205,206 is the outermost layer, the numbers also provide an indication of how many layers 203,204,205,206 remain on the stack.

In other embodiments, rather than numbering the tabs 217,218,219,220, other indication systems can be used. For example, in one embodiment the tabs 217,218,219,220 are color-coded. The color codes may go, for example from varying shades of green to varying shades of red so as to mimic the indications of a traffic light. In another embodiment, letters can be used rather thn the numbers in the preceding paragraph. For example, tab 217 may have the letter “A” disposed thereon, while tab 218 has the letter “B” disposed thereon, and so forth. The order of the letters could be reversed as well, with the letter “J” disposed on tab 217, the number “I” disposed on tab 218, and so forth. Other identification systems will be obvious to those of ordinary skill in the art having the benefit of this disclosure.

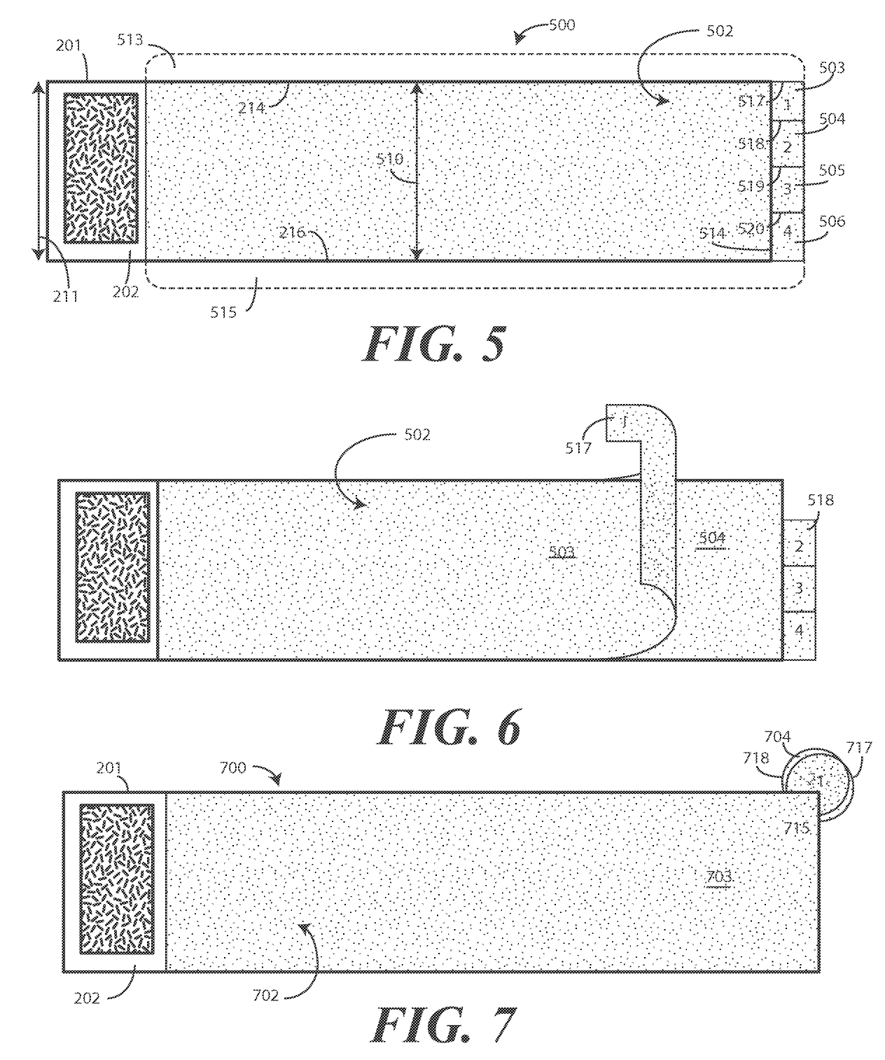

In one or more embodiments, peeling away a layer not only reveals the next lower layer, it also can reveal the next tab as well. As shown in FIG. 4, when layer 203 is peeled away from the stack by tab 217, this reveals the next uppermost tab, which is tab 218 in this embodiment. This reveal is optional, as other embodiments will have all tabs exposed, as will be described below with reference to FIGS. 5 and 6.

It should also be noted that the tabs may be configured in any of a number of ways. Turning now to FIGS. 5 and 6, illustrated therein is another liner system 500 configured in accordance with one or more embodiments of the disclosure. The liner system 500 of FIGS. 5 and 6 is again being used with a blood pressure cuff 201. The liner system 500 is attached to the interior side 502 of the blood pressure cuff 201 is shown in FIG. 5. When the blood pressure cuff 201 is wrapped about the arm of a patient, the liners 503,504,505,506 of the liner system 500 prevent the blood pressure cuff 201 from coming in contact with the user's arm.

As with the embodiment of FIGS. 2-4, the liner system 500 includes a plurality of layers 503,504,505,506 of material, arranged in a stack 502. The stack 502 of FIG. 5 has a minor dimension 510 that is approximately equal to the width 211 of the blood pressure cuff 201 in this illustrative embodiment. However, as shown by the dashed lines of FIG. 5, the minor dimension 510 could be greater than the width 211 of the blood pressure cuff 201 as well. For example, the top 513 of the liner system 500 can extend beyond the top edge 214 of the blood pressure cuff 201. Alternatively, the bottom 515 of the liner system 500 can extend beyond the bottom edge 216 of the blood pressure cuff 201 as well. Of course, combinations of the two can be used as well. The dashed lines illustrate alternative configurations to the solid line embodiment shown in FIGS. 5 and 6.

In one embodiment, each layer 503,504,505,506 of material is peelable from other layers of material such that an uppermost layer, e.g., layer 503, of the stack can be peeled from the stack as shown in FIG. 6 after use to reveal a next uppermost layer, e.g., layer 504, of the stack. To make peeling easier, each layer 503,504,505,506 of the stack also includes a tab 517,518,519,520 to facilitate peeling the each layer 503,504,505,506 of material from the stack 502.

As with the embodiment shown in FIGS. 2-4, in FIGS. 5 and 6 each tab 517,518,519,520 is staggered from other tabs of the stack 502. However, in contrast to the embodiment of FIGS. 2-4, where the tabs (217,218,219,220) were disposed along a major side of the blood pressure cuff 201, i.e., they extended vertically above the top 214 of the blood pressure cuff 201, the tabs 517,518,519,520 of the liner system 500 of FIG. 5 are disposed along a minor side of the blood pressure cuff 201 in that they extend beyond a side edge 514 of the blood pressure cuff 201. Each tab 517,518,519,520 is staggered from the next, with the staggering occurring vertically in this embodiment. This results in each layer 503,504,505,06 of material having different dimensions than other layers of material in the stack 502. Additionally, each tab 517,518,519,520 is numbered, although other indication systems, such as color-coding, can be used as well as noted above.

In the embodiment of FIGS. 5 and 6, the tabs 517,518,519,520 are arranged such that the uppermost layer of the stack 502, which is layer 503 in this embodiment, comprises the uppermost tab, i.e., tab 517. Sequentially next lower layers contain sequentially next lower tabs in this embodiment. For example, layer 504 has tab 518, which is lower than tab 517 of layer 503.

In the embodiment of FIGS. 5 and 6, there are five or fewer layers. As noted above, testing has shown that a smaller number of layers can be preferable when the blood pressure cuff 201 is disposable, while a lesser number of layers can be advantageous when the blood pressure cuff 201 is permanent or reusable.

To illustrate the flexibility offered by embodiments of the disclosure, FIG. 7 illustrates yet another staggered tab arrangement. In the embodiment of FIG. 7, a liner system 700 is attached to an interior side 202 of a blood pressure cuff 201. When the blood pressure cuff 201 is wrapped about the arm of a patient, the liner system 700 prevents the blood pressure cuff 201 from coming in contact with the user's arm.

The liner system 700 includes a plurality of layers 703,704 of material, arranged in a stack 702. As with previous embodiments, the minor dimension of the stack 702 can be equal to, or greater than, the width of the blood pressure cuff 201, with one or more of the top of the stack 702 extending beyond the top of the blood pressure cuff 201, the bottom of the stack extending beyond the blood pressure cuff 201, or combinations thereof. Alternatively, the minor dimension can be approximately equal to the width of the blood pressure cuff.

As with previous embodiments, each layer 703,704 of material is peelable from other layers of material such that an uppermost layer, e.g., layer 703, of the stack 702 can be peeled from the stack 702 after use to reveal a next uppermost layer, e.g., layer 704, of the stack. To make peeling easier, each layer 703,704 of the stack also includes a tab 717,718. The tabs 717,718 can be staggered relative to each other, or alternatively can be disposed atop each other.

In this illustrative embodiment, each tab 717,718 is round. Each tab 717,718 is also numbered. The tabs 717,718 of this embodiment extend distally away from their corresponding layers 703,704 at the corner 715 of the blood pressure cuff 201. This corner alignment makes the tabs 717,718 more graspable as the blood pressure cuff is initially removed from the patient's arm. Other locations at which to dispose the tabs 717,718 will be obvious to those of ordinary skill in the art having the benefit of this disclosure.

Turning now to FIGS. 8 and 9, illustrated therein is another liner system 800 configured in accordance with one or more embodiments of the disclosure. The liner system 800 includes a plurality of layers 803,804,805,806 of material, arranged in a stack 802. Each layer 803,804,805,806 of material is peelable from other layers of material such that an uppermost layer of the stack 802 can be peeled from the stack 802 after use to reveal a next uppermost layer of the stack 802. To make peeling easier, each layer 803,804,805,806 of the stack 802 also includes a tab 817,818,819,820. The tabs 817,818,819,820 can be staggered relative to each other, or alternatively can be disposed atop each other.

In this embodiment, each layer comprises a writable surface 821 to receive writing 822 from a writing instrument 823. Accordingly, a healthcare services provider can write, for example, a patient's name on the writable surface 821. In this case, the patient's name is Buster, and Buster's name has been written on the writable surface 821 with the writing instrument 823. Providing the writable surface 821 serves multiple functions. First, it assures the patient that the uppermost layer has not been used with another patient. The patient can watch their name being written on the writable surface 821, and sees that no other name has been written thereon. Second, it provides confirmation to the healthcare services provider that the uppermost layer has not been used either. If any dirt, debris, writing, or other matter was present on the writing surface 821, the healthcare services provider could simply remove the uppermost layer to reveal the next uppermost layer. Additionally, it provides a reminder to the healthcare services provider to remove the uppermost layer after use. By seeing Buster's name on the writable surface 821, the healthcare services provider is reminded to remove the uppermost layer to leave a clean writable surface for the next user. Finally, the writable surface 821 provides a way to personalize an otherwise unpersonable procedure, namely, having blood pressure taken.

In one embodiment, the writable surface 821 is opaque. For example, the writable surface 821 may be a matte white surface. A person may apply writing 822, for example, with an ink pen. In other embodiments, the writable surface 821 can be color-coded. For example, embodiments of the disclosure contemplate that the liner system 800 can be manufactured in different sizes to accommodate different size blood pressure cuffs. Accordingly, color-coding the writable surface 821 provides a quick and easy indication to the healthcare services provider as to what size the blood pressure cuff is.

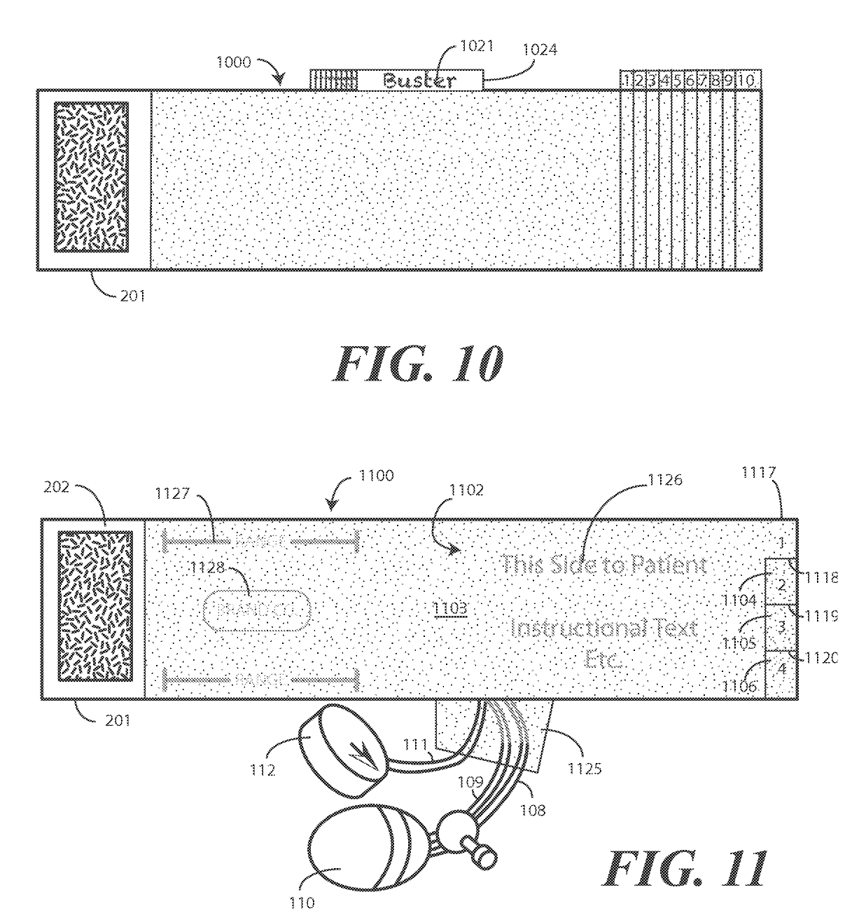

In the illustrative embodiment of FIGS. 8 and 9, the writable surface 821 is disposed along a major face of the liner system 800 interior of the edges of the blood pressure cuff 201. However, it could be placed in other locations as well. Turning now to FIG. 10, illustrated therein is another liner system 1000 where the writable surface 1021 is disposed on a tab 1024 extending from a major side of the blood pressure cuff 201. This location moves the writable surface 1021 away from portions of the blood pressure cuff 201 that inflate when systolic and diastolic blood pressure measurements are taken. This is just one alternative location for the writable surface 1021. Others will be obvious to those of ordinary skill in the art having the benefit of this disclosure.

Turning now to FIG. 11, illustrated therein is yet another liner system 1100 configured in accordance with one or more embodiments of the disclosure. The liner system 1100 includes a plurality of layers 1103,1104,1105,1106 of material, arranged in a stack 1102. Each layer 1103,1104,1105,1106 of material is peelable from other layers of material such that an uppermost layer of the stack 1102 can be peeled from the stack 1102 after use to reveal a next uppermost layer of the stack 1102. To make peeling easier, each layer 1103,1104,1105,1106 of the stack 1102 also includes a tab 1117,1118,1119,1120. The tabs 1117,1118,1119,1120 can be staggered relative to each other, or alternatively can be disposed atop each other.

In this illustrative embodiment, each layer 1103,1104,1105,1106 is pellucid such that instructional indicia 1126, sizing indicia 1127, branding indicia 1128 or other information disposed along an inner side 202 of the blood pressure cuff 201 can be seen through the stack 1102. In other embodiments, the layers 1102,1104,1105,1106 can be opaque and/or color-coded.

In the embodiment of FIG. 11, the each layer 1103,1104,1105,1106 of material defines a peninsular extension 1125 to cover at least a portion of one or more tubes 108,109,111 extending outwardly from the blood pressure cuff 201. For example, where one or more tubes 108,109 extend to an inflation source 110, the peninsular extension 1125 can cover at least a portion of these tubes 108,109 to ensure that they do not contact a patient when the blood pressure cuff 201 is wrapped about the patient's arm and inflated. Similarly, where the one or more tubes include a tube 111 extending to a sphygmomanometer 112, the peninsular extension 1125 can cover at least a portion of this tube 111 to ensure that it does not contact a patient when the blood pressure cuff 201 is wrapped about the patient's arm and inflated.

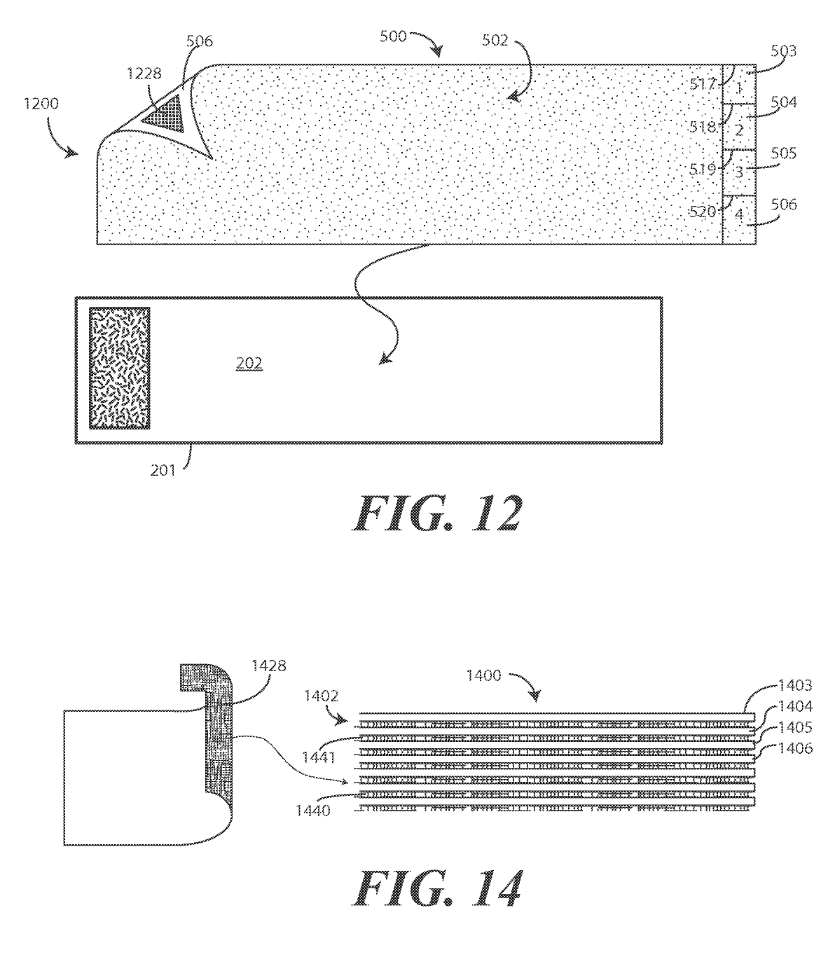

Turning now to FIG. 12, illustrated therein is a system 1200 configured in accordance with one or more embodiments of the disclosure. The system 1200 includes a blood pressure cuff 201 and a liner system 500. The liner system 500 includes a plurality of layers 503,504,505,506 of material, arranged in a stack 502. Each layer 503,504,505,506 of material is peelable from other layers of material such that an uppermost layer of the stack 502 can be peeled from the stack 502 after use to reveal a next uppermost layer of the stack 502. To make peeling easier, each layer 503,504,505,506 of the stack 502 also includes a tab 517,518,519,520.

In this embodiment, the stack 502 can be ordered as an accessory item and attached to an inner side 202 of the blood pressure cuff 201. In one embodiment, the lowermost layer, i.e., layer 506, of the stack 502 comprises a fixative 1228 to attach the stack 502 to the inner surface 202 of the blood pressure cuff 202. The fixative 1228 can take any of a variety of forms.

In one embodiment, the fixative 1228 is an adhesive. Adhesives work well when the stack 502 comprises layers 503,504,505,506 that are disposable. In another embodiment, the fixative 1228 is a hook and loop fastener. Hook and loop fasteners work well when the stack 502 comprises layers 503,504,505,506 that are reusable. In yet another embodiment, the fixative 1228 is an electrostatic surface that adheres to the inner surface 202 of the blood pressure cuff 201. Other fixatives 1228 will be obvious to those of ordinary skill in the art having the benefit of this disclosure.

With the fixative 1228 disposed along the lowermost layer, the stack 500 can simply be attached to the inner surface 202 of the blood pressure cuff 201. When the layers 503,504,505,506 are depleted, another stack 500 can be obtained and attached to the inner surface of the blood pressure cuff 201.

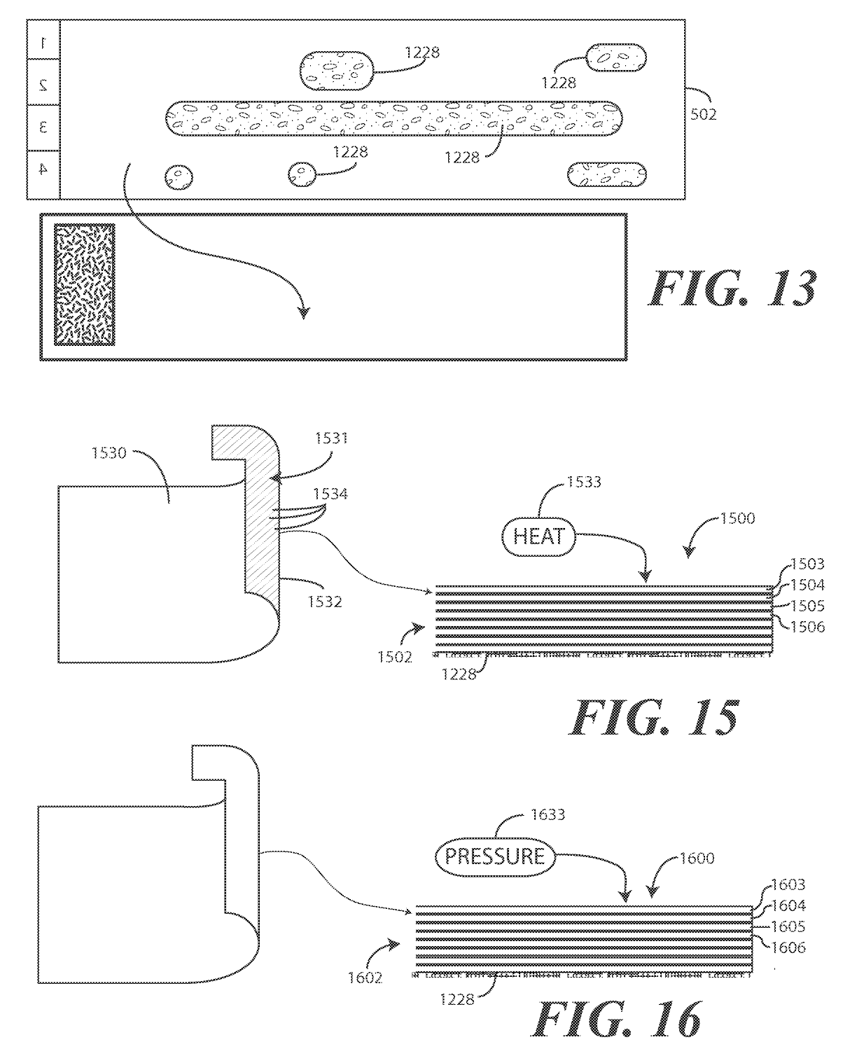

As shown in FIG. 13, in one or more embodiments the fixative 1228 can be selectively disposed along the lowermost layer of the stack 502. “Selectively disposing” the fixative 1228 along the lowermost layer of the stack 502 refers to placing the fixative 1228 along some portions of the lowermost layer, and not placing the fixative 1228 along other portions of the lowermost layer. For example, in one embodiment the fixative 1228 may only be placed in the corners of the lowermost layer. In other embodiments, the fixative 1228 may be placed along the lowermost layer in a checkerboard pattern, and so forth. Selective disposition of the fixative 1228 can advantageously be used in one or more embodiments to prevent layer shifting. Other methods of selectively disposing the fixative 1228 along the lowermost layer will be obvious to those of ordinary skill in the art having the benefit of this disclosure.

Turning now to FIG. 14, illustrated therein is another liner system 1400 configured in accordance with one or more embodiments of the disclosure. As noted in the embodiments above, liner systems in accordance with embodiments of the disclosure include a plurality of layers 1403,1404,1405,1406, arranged in a stack 1402 that are peelable from other layers such that an uppermost layer of the stack 1402 can be peeled from the stack after use to reveal a next uppermost layer of the stack 1402.

In one embodiment, each of the layers 1403,1404,1405,1406 is manufactured from a plastic sheet material. In one embodiment, each of the layers 1403,1404,1405,1406 is manufactured from vinyl. In yet another embodiment, each of the layers 1403,1404,1405,1406 is manufactured from polyester. Other materials will be obvious to those of ordinary skill in the art having the benefit of this disclosure.

In one embodiment, the plastic sheet material or vinyl can simply be electrostatically coupled together such that each of the layers 1403,1404,1405,1406 is held to the other layers by an electrostatic charge. Accordingly, the layers 1403,1404,1405,1406 would be electrostatically coupled together.

In another embodiment, a releasable fixative 1428 can be disposed between each of the layers 1403,1404,1405,1406. For example, the releasable fixative 1428 can be a releasable adhesive that allows each layer 1403,1404,1405,1406 to be peeled from the other layer and/or the lowermost layer to be peeled from a blood pressure cuff. Accordingly, the layers 1403,1404,1405,1406 would be adhesively coupled together.

In one or more embodiments, the releasable adhesive can have different adhesion based upon its location in the stack 1402. For example, where an adhesive layer 1440 is disposed lower in the stack 1402 than another adhesive layer 1441, testing has shown it can be advantageous to make this adhesive layer 1440 stronger than the other adhesive layer 1441. Accordingly, while it takes more and more force to peel lower layers away from the stack than higher layers, overall reliability can be enhanced by making lower adhesive layers 1440 “stickier” than higher adhesive layers 1441. In one embodiment, adhesive strength can be increased by selectively depositing adhesive, e.g., as a series of dots along the adhesive layer 1440, with varying spatial separation between dots. Lower layers could have a tighter spacing, i.e., a more densely patterned adhesive layer 1440, while upper layers could be less densely patterned with more space between adhesive depositions. This varied adhesive patterning could help to ensure that layers are removed serially in order. In other embodiments, all adhesive layers can have equal stickiness.

In another embodiment, which will be described in more detail below with reference to FIG. 15, the releasable fixative 1428 may be a polyester coating disposed along each layer 1403,1404,1405,1406. Alternatively, when the layers 1403,1404,1405,1406 are manufactured from polyester, the releasable fixative 1428 can be a surface texture along one side of the layer. For example, in one embodiment the layers 1403,1404,1405,1406 are manufactured from polyester and one side is made to be fibrous. This fibrous surface functions as a releasable fixative 1428 after the application of heat. Thus, when a polyester coating is used, or alternatively when a side of a polyester layer is textured, heat may be applied to make the layers 1403,1404,1405,1406 stick to each other. The seal formed, especially when a fibrous polyester coating and/or surface is used as the releasable fixative 1428, is easily broken such that the layers 1403,1404,1405,1406 can be peeled away from each other. This method results in the layers 1403,1404,1405,1406 being thermally coupled together. The releasable fixatives 1428 of adhesive coupling, thermal coupling, electrostatic coupling, or even pressure coupling can be advantageous when the layers 1403,1404,1405,1406 are disposable.

In other embodiments, such as when the layers 1403,1404,1405,1406 are reusable, the releasable fixative 1428 can be of a different type. For example, in one embodiment the releasable fixative 1428 is a hook and loop fastener. In another embodiment, the releasable fixative 1428 is a releasable textile adhesive. Still other releasable adhesives suitable for use with embodiments of the disclosure will be obvious to those of ordinary skill in the art having the benefit of this disclosure.

Turning now to FIG. 15, illustrated therein is another liner system 1500 configured in accordance with one or more embodiments of the disclosure. The liner system 1500 includes a plurality of layers 1503,1504,1505,1506 arranged in a stack 1502. Each layer 1503,1504,1505,1506 is peelable from other layers such that an uppermost layer of the stack 1502 can be peeled from the stack after use to reveal a next uppermost layer of the stack 1502.

Each layer 1503,1504,1505,1506 defines a first major face 1530 and a second major face 1532. In one embodiment, a fibrous coating/surface 1531 is disposed along one of the first major face 1530 or the second major face 1532. In this illustrative embodiment, the fibrous coating/surface 1531 is disposed along the second major face 1532.

In one embodiment, the fibrous coating/surface 1532 comprises a polyester coating that is applied to the plastic material defining each layer 1503,1504,1505,1506. In another embodiment, the fibrous coating/surface or alternatively is a textured, fibrous surface defined across each layer 1503,1504,1505,1506 when each layer 1503,1504,1505,1506 is manufactured from a polyester-based material. Heat 1533 can then be applied to the stack 1502 such that the fibers 1534 of the fibrous coating/surface 1532 thermally couple to the first major face of the next lowermost layer. Said differently, when the fibrous coating/surface 1532 comprises a polyester coating or a textured, fibrous surface in a polyester-based material layer, the plurality of layers 1503,1504,1505,1506 of material can be thermally coupled together only along fibers of the polyester coating/surface 1532. This leads to a simple, releasable fixative that is not sticky when the layers 1503,1504,1505,1506 are peeled apart. In one embodiment, even when thermal coupling is used for the layers 1503,1504,1505,1506, the lowermost layer may still include a fixative 1228 to attach the stack 1502 to a blood pressure cuff (201).

By contrast to using either adhesive or thermal coupling, the liner system 1600 of FIG. 16 uses pressure to couple the various layers 1603,1604,1605,1606. Testing has shown that when the layers 1603,1604,1605,1606 are manufactured from a thermoplastic material, pressure 1633 can be used to releasably adhere the layers 1603,1604,1605,1606 together. Specifically, once the layers 1603,1604,1605,1606 are arranged into a stack 1602, pressure 1633 can be applied to the stack 1602. This pressure causes the layers 1603,1604,1605,1606 to stick together, yet be peelable from each other. In one embodiment, even when pressure coupling is used for the layers 1603,1604,1605,1606, the lowermost layer may still include a fixative 1228 to attach the stack 1502 to a blood pressure cuff (201).

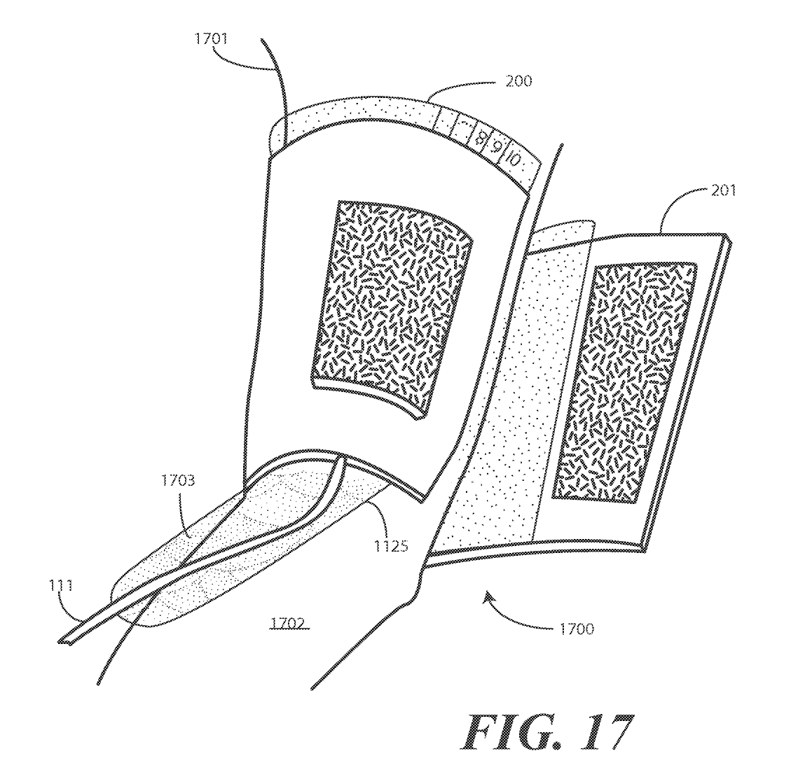

Turning now to FIG. 17, illustrated therein is one embodiment of a system 1700 being used on a patient 1701. As shown, a blood pressure cuff 201 has attached thereto along an inner surface 202 a liner system 200. The liner system 200 includes a plurality of layers of material, arranged in a stack. Each layer of material is peelable from other layers of material such that an uppermost layer of the stack can be peeled from the stack after use to reveal a next uppermost layer of the stack. As shown, the liner system prevents the inner surface 202 of the blood pressure cuff 201 from coming in contact with the user's arm 1702. Moreover, the peninsular extension 1125 precludes portions of tubing 111 extending from the blood pressure cuff 201 from contacting the user's arm 1702 as well. In this embodiment, the peninsular extension 1125 is extended in length and has a releasable adhesive 1703 to attach to the tubing 111.

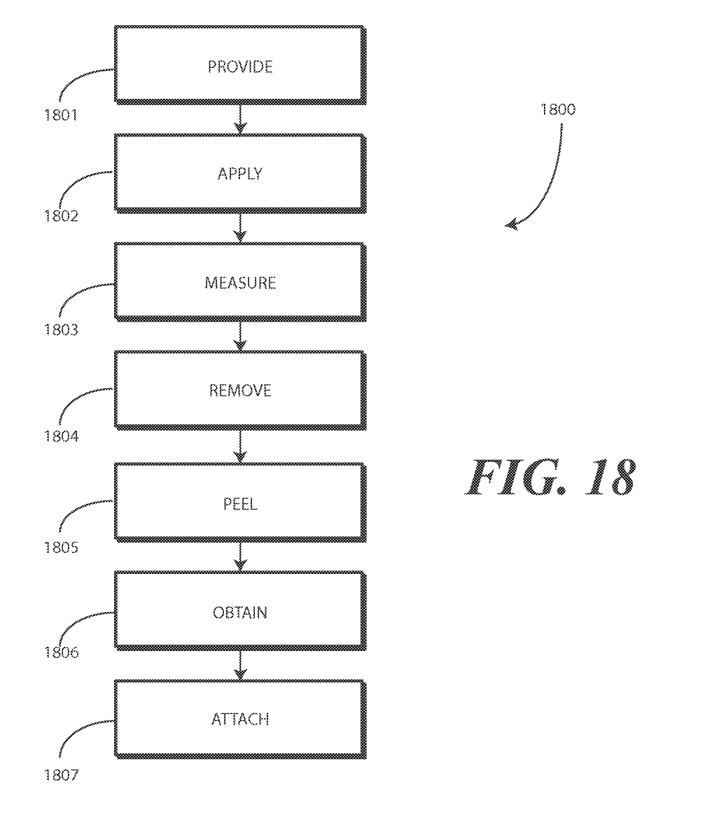

Turning now to FIG. 18, illustrated therein is a method 1800 for using a liner system in accordance with one or more embodiments of the disclosure. At step 1801, the method 1800 provides a blood pressure cuff. In one embodiment, the blood pressure cuff is coupled to a plurality of layers of material. In one embodiment, the layers of material are arranged in a stack. In one embodiment, the layers are held together by a fixative. The fixative can be an adhesive. Alternatively, the fixative can be an electrostatic or pressure coupling between the layers. Where the layers are reusable, the layers can be hook and loop fasteners, snaps, and so forth. In one embodiment, each layer of material is peelable from other layers of material such that an uppermost layer of the stack can be peeled from the stack after use to reveal a next uppermost layer of the stack.

At step 1802, the method 1800 applies the blood pressure cuff about a patient's arm with the uppermost layer of the stack disposed adjacent with the patient's arm. Said differently, at step 1802, the method 1800 applies the blood pressure cuff to the patient's arm with the stack disposed between the cuff and the arm, thereby preventing contact between the arm and the cuff.

At step 1803, the method 1800 optionally takes the patient's systolic and diastolic blood pressure. At step 1804, the method removes the blood pressure cuff from the patient's arm.

To ensure that no cross-contamination between patients occurs, at step 1805, the method 1800 peels the uppermost layer of the stack to reveal the next uppermost layer of the stack. This ensures that any contaminants, bacteria, viruses, or pathogens disposed along the uppermost layer due to the usage of the blood pressure cuff are removed before the cuff is used on the next patient.

In one embodiment, after step 1805, the blood pressure cuff is discarded. However, in other embodiments, a new stack of liners can be obtained. In this latter embodiment, step 1805 can include removing the lowermost layer of the stack from the blood pressure cuff. Where the blood pressure cuff is intended to be reusable, the method 1800 can optionally include, at step 1806, obtaining another plurality of layers of material, arranged in another stack. Optional step 1806 can then include attaching the another stack to an interior of the blood pressure cuff. The method 1800 can then repeat, beginning anew at step 1801.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A liner system for use with a blood pressure cuff, the liner system comprising:
a plurality of layers of material, arranged in a stack, each layer of material:
peelable from other layers of material such that an uppermost layer of the stack can be peeled from the stack after use to reveal a next uppermost layer of the stack; and
comprising a tab to facilitate peeling the each layer of material from the stack, each tab being staggered from other tabs of the stack;
a lowermost layer of the stack comprising a fixative to attach the stack to an inner surface of the blood pressure cuff.

2. The liner system of claim 1, the fixative comprising an adhesive, the each layer of material disposable.

3. The liner system of claim 2, the adhesive selectively disposed along the lowermost layer.

4. The liner system of claim 1, the each layer of material defining a major dimension less than a length of the blood pressure cuff and a minor dimension greater than or equal to a width the blood pressure cuff to preclude contact of the blood pressure cuff with a patient's arm.

5. The liner system of claim 1, the plurality of layers of material one of adhesively coupled together to define the stack or electrostatically coupled together to define the stack.

6. The liner system of claim 1, the plurality of layers of material thermally coupled together to define the stack.

7. The liner system of claim 6, the each layer of material defining a first major face and a second major face, further comprising a fibrous coating disposed along, or a fibrous surface defined along, one of the first major face or the second major face.

8. The liner system of claim 7, the each layer of material comprising a polyester-based material with the fibrous surface defined along the one of the first major face or the second major face, the plurality of layers of material thermally coupled together only along fibers of the fibrous surface.

9. The liner system of claim 1, the tab and the other tabs disposed along a minor side of the stack.

10. The liner system of claim 1, the tab and the other tabs disposed along a major side of the stack.

11. The liner system of claim 1, the tab and the other tabs numbered.

12. The liner system of claim 1, the tab and the other tabs arranged such that the uppermost layer of the stack comprising an uppermost tab can be peeled from the stack after use to reveal a next uppermost tab.

13. The liner system of claim 1, the each layer of material having different dimensions than other layers of material in the stack.

14. The liner system of claim 1, the each layer comprising a writable surface to receive writing from a writing instrument.

15. The liner system of claim 1, the stack comprising ten or fewer layers.

16. The liner system of claim 1, further comprising the blood pressure cuff, the stack attached to the inner surface of the blood pressure cuff.

17. The liner system of claim 16, the tab and the other tabs extending distally from beyond an edge of the blood pressure cuff.

18. The liner system of claim 16, further comprising a sphygmomanometer operable with the blood pressure cuff and coupled to the blood pressure cuff by one or more tubes, the each layer of material defining a peninsular extension to cover at least a portion of the one or more tubes.

19. A method of using a liner system, the method comprising:
providing a blood pressure cuff coupled to a plurality of layers of material, arranged in a stack, by a fixative, each layer of material peelable from other layers of material such that an uppermost layer of the stack can be peeled from the stack after use to reveal a next uppermost layer of the stack;
applying the blood pressure cuff about a patient's arm with the uppermost layer of the stack disposed adjacent with the patient's arm;
removing the blood pressure cuff from the patient's arm; and
peeling the uppermost layer of the stack to reveal the next uppermost layer of the stack.

20. The method of claim 19, further comprising:
removing a lowermost layer of the stack from the blood pressure cuff;
obtaining another plurality of layers of material, arranged in another stack; and
attaching the another stack to an interior of the blood pressure cuff.
